(19) United States
(12) Patent Application Publication
(10) Pub. No.: US 2021/0032372 A1
(43) Pub. Date: Feb. 4, 2021

(54) METHOD FOR PRODUCING CELLULOSE PARTICLES OR CELLULOSE ACETATE PARTICLES

(71) Applicant: JNC CORPORATION, Tokyo (JP)

(72) Inventors: Yasutaka IWASA, Kumamoto (JP); Takeshi KUROKAWA, Kumamoto (JP); Satoru MAEDA, Kumamoto (JP); Junji TANOUE, Kumamoto (JP); Shinichi Furusato, Kumamoto (JP)

(73) Assignee: JNC CORPORATION, Tokyo (JP)

(21) Appl. No.: 16/923,076

(22) Filed: Jul. 7, 2020

(30) Foreign Application Priority Data
Jul. 30, 2019 (JP) 2019-139922

(51) Int. Cl. C08B 3/06; A61K 8/73

(52) U.S. Cl. CPC C08B 3/06; A61K 8/731

(57) ABSTRACT

Provided is a method for producing cellulose particles or cellulose acetate particles. By a production method including: (a) dissolving cellulose acetate in an organic solvent and preparing a cellulose acetate solution; (b) obtaining an emulsion by passing the cellulose acetate solution and an aqueous medium through a gap between an outer cylinder and an inner cylinder arranged coaxially inside the outer cylinder and rotating at least one of the outer cylinder and the inner cylinder; and (c) precipitating cellulose acetate particles from the emulsion, cellulose acetate particles are produced. By further saponifying the cellulose acetate obtained by the production method, cellulose particles are produced.

### CROSS-REFERENCE TO RELATED APPLICATION

This application claims the priority benefit of Japan Application No. 2019-139922, filed on Jul. 30, 2019. The entirety of the above-mentioned patent application is hereby incorporated by reference herein and made a part of this specification.

### BACKGROUND

### Technical Field

The disclosure relates to a method for producing cellulose particles or cellulose acetate particles.

### Related Art

Cellulose particles have resistance to an acidic solvent and a basic solvent, and can be modified to allow addition of various substituents thereto. Hence, the cellulose particles have been used as an adsorbent for various substances in a wide range of fields such as separation, purification and desalination of various substances and so on. Examples of the fields where the cellulose particles are used include a gel filtration method (a method for fractionating substances depending on a difference in molecule size). The gel filtration method is applicable to both an aqueous solution and an organic solvent, and also applicable to a compound having any molecular weight. Hence, it has been widely used not only on a laboratory scale but also on an industrial scale (Patent Document 1).

In addition, since the cellulose particles have excellent adsorption properties and relatively large mechanical strength, their application to an industrially applicable adsorbent for antibody drug purification (Patent Document 2) or an adsorbent for a virus such as influenza or the like (Patent Document 3) has also received attention.

The cellulose particles have also been widely used as a powder to be blended with a cosmetic.

Conventionally, a method for producing cellulose particles has been carried out using cellulose or cellulose acetate as a raw material and various solvents and so on (Patent Documents 4 to 8). In recent years, there has also been proposed a method for efficiently producing cellulose particles without using a harmful substance such as chlorinated hydrocarbons or the like (Patent Document 9).

By the way, there is an emulsification method in which an emulsion is obtained by, while passing a mixture of a disperse phase and a continuous phase through a gap between an outer cylinder and an inner cylinder that are arranged coaxially, rotating the inner cylinder at high speed in the outer cylinder (Patent Documents 10 to 11). In this method, a laminar flow state with no eddies is formed in the mixture passing through the gap between the two rotating cylinders, and a strong shearing force and a uniform reaction field are provided. According to this emulsification method, it is known that continuously uniform emulsified particles can be obtained. However, there is no disclosure regarding its application to a method for producing cellulose particles.

### PATENT DOCUMENTS

[Patent Document 1] Japanese Patent Laid-open No. S56-24430

[Patent Document 2] WO 08/146906

[Patent Document 3] Japanese Patent Laid-open No. 2011-220992

[Patent Document 4] Japanese Patent Laid-open No. S55-44312

[Patent Document 4] Japanese Patent Laid-open No. H6-254373

[Patent Document 6] Japanese Patent Laid-open No. 2012-87202

[Patent Document 7] Japanese Patent Laid-open No. H1-277570

[Patent Document 8] Japanese Patent Laid-open No. S55-40618

[Patent Document 9] WO 2015/029790

[Patent Document 10] Japanese Patent Laid-open No. H04-187227

[Patent Document 11] Japanese Patent Laid-open No. 2005-66392

Cellulose particles are used in various fields, and biodegradable cellulose tends to be favored due to recent nature-conscious trends. Thus, they are in great demand. However, in a conventional production method, control of the particle size requires ingenuity, and ultrafiltration or the like in a step of washing the cellulose particles after formation thereof may take time, or a classification step may be required to obtain monodisperse particles. Therefore, it is hard to say that productivity is sufficient, and there is room for improvement.

The disclosure provides a method for producing cellulose particles or cellulose acetate particles, capable of controlling particle size and particle size distribution and increasing productivity.

### SUMMARY

As a result of earnest studies, the present inventors found that cellulose particles or cellulose acetate particles can be efficiently produced by an emulsification method performed by cylinder rotation in which at least one of an outer cylinder and an inner cylinder is rotated, thereby accomplishing the disclosure.

That is, the disclosure includes the following aspects.

[1] A method for producing cellulose acetate particles, including:

(a) dissolving cellulose acetate in an organic solvent and preparing a cellulose acetate solution;

(b) obtaining an emulsion by passing the cellulose acetate solution and an aqueous medium through a gap between an outer cylinder and an inner cylinder arranged coaxially inside the outer cylinder and rotating at least one of the outer cylinder and the inner cylinder; and

(c) precipitating cellulose acetate particles from the emulsion.

[2] The method for producing cellulose acetate particles as described in [1], wherein in (a), the organic solvent is ethyl acetate, a mixed solvent of ethyl acetate and acetone, or cyclohexanone.

[3] The method for producing cellulose acetate particles as described in [1] or [2], wherein in (a), the cellulose acetate is cellulose diacetate having a degree of acetylation of 45% to 57%.

[4] The method for producing cellulose acetate particles as described in any one of [1] to [3], wherein in (c), the cellulose acetate particles are precipitated by cooling of the emulsion and/or addition of a poor solvent to the emulsion.

[5] The method for producing cellulose acetate particles as described in [4], wherein the poor solvent is water, alcohols, glycols, esters, or a mixture thereof.

[6] The method for producing cellulose acetate particles as described in any one of [1] to [5], wherein in (b), the passage through the gap between the outer cylinder and the inner cylinder is performed a plurality of times.

[7] The method for producing cellulose acetate particles as described in any one of [1] to [6], wherein in (b), a particle size of the cellulose acetate particles is controlled by changing a rotational speed of the outer cylinder and/or the inner cylinder.

[8] The method for producing cellulose acetate particles as described in any one of [1] to [7], wherein in (b), the aqueous medium is water, an ethyl acetate-containing aqueous solution, or a cyclohexanone-containing aqueous solution.

[9] The method for producing cellulose acetate particles as described in any one of [1] to [8], wherein in (a), the cellulose acetate solution is prepared so as to contain 1% by weight to 30% by weight of cellulose acetate with respect to the whole of the cellulose acetate solution.

[10] A method for producing cellulose particles, including:

producing cellulose acetate particles by the method for producing cellulose acetate particles as described in any one of [1] to [9]; and

(d) saponifying the cellulose acetate particles.

[11] Cellulose acetate particles produced by the method as described in any one of [1] to [9].

[12] A chromatography packing material, containing the cellulose acetate particles as described in [11], wherein the cellulose acetate particles are modified or unmodified.

[13] A cosmetic, containing the cellulose acetate particles as described in [11], wherein the cellulose acetate particles may be modified.

[14] A daily commodity, containing the cellulose acetate particles as described in [11], wherein the cellulose acetate particles are modified or unmodified.

[15] Cellulose particles produced by the method as described in [10].

[16] A chromatography packing material, containing the cellulose particles as described in [15], wherein the cellulose particles are modified or unmodified.

[17] A cosmetic, containing the cellulose particles as described in [15], wherein the cellulose particles are modified or unmodified.

[18] A daily commodity, containing the cellulose particles as described in [15], wherein the cellulose particles are modified or unmodified.

According to the disclosure, cellulose particles or cellulose acetate particles can be produced in a manner enabling control of particle size and particle size distribution and increasing the productivity.

### BRIEF DESCRIPTION OF THE DRAWINGS

FIG. 1 is a schematic view of a device that performs a step (b).

### DESCRIPTION OF THE EMBODIMENTS

Hereinafter, an embodiment of the disclosure will be described in detail.

A method for producing cellulose acetate particles of the disclosure includes the following steps (a) to (c) in this order:

(a) dissolving cellulose acetate in an organic solvent and preparing a cellulose acetate solution;

(b) obtaining an emulsion by passing the cellulose acetate solution and an aqueous medium through a gap between an outer cylinder and an inner cylinder arranged coaxially inside the outer cylinder and rotating at least one of the outer cylinder and the inner cylinder; and

(c) precipitating cellulose acetate particles from the emulsion.

A method for producing cellulose particles of the disclosure includes (d) saponifying the cellulose acetate particles produced by the method for producing cellulose acetate particles of the disclosure.

Hereinafter, the above steps will be described in order.

In this specification, when described as “cellulose (acetate) particles,” it may mean cellulose acetate particles or cellulose particles.

[Step (a)]

In (a), cellulose acetate as a raw material is dissolved in an organic solvent and a cellulose acetate solution is prepared.

The cellulose acetate is a semisynthetic polymer obtained by acetic acid esterification of cellulose being a natural polymer. The cellulose acetate that has been industrially widely used is roughly classified into two types, cellulose diacetate and cellulose triacetate, whose degrees of acetylation are generally about 50% to 57% and about 60% to 62%, respectively.

In the method of the disclosure, either of cellulose diacetate and cellulose triacetate may be used as the raw material, and cellulose diacetate is preferably used. The cellulose diacetate used in the disclosure is not particularly limited as long as it can be generally defined as cellulose diacetate, and its degree of acetylation is preferably 45% to 57%, more preferably 53% to 56%. The cellulose diacetate can be dissolved in more kinds of solvents by having a degree of acetylation of 45% to 57%.

Specifically, the cellulose diacetate obtained by acetylation of linter pulp, wood pulp or the like with acetic acid and/or acetic anhydride followed by partial saponification can be used. In this case, the degree of esterification can be appropriately adjusted so that the degree of acetylation falls in the above range. Please refer to, for example, Japanese Patent Laid-Open No. S62-000501.

The organic solvent that dissolves the cellulose acetate is not particularly limited as long as being capable of dissolving cellulose acetate, and a solvent having low toxicity is preferable. In addition, one having a lower boiling point than water is preferable. In the case of using cellulose diacetate as a raw material, a wider range of solvents can be used than in the case of using cellulose triacetate, and among them, a solvent having low toxicity can be selected for use.

As the organic solvent, one kind or a mixture of two or more kinds of organic solvents may be used as long as the obtained cellulose acetate solution forms an aqueous medium and an emulsion to be described later. Specific examples thereof include an organic solvent, such as acetic acid, acetone, dimethylformamide, dimethyl sulfoxide, cyclohexanone, ethyl acetate, butyl acetate or the like, and a mixture of these organic solvents. For example, acetic acid, acetone or the like, which is compatible with an aqueous medium, is used as a mixture together with a solvent that does not mix with an aqueous medium. Particularly preferable examples among the above include ethyl acetate, a mixed solvent of ethyl acetate and acetone, and cyclohexanone. When a mixed solvent of ethyl acetate and acetone is used, a mixing ratio (weight ratio) of ethyl acetate to acetone is preferably 99.9:0.1 to 60:40.

As described above, according to the disclosure, since it is possible to use a solvent having low toxicity, cellulose (acetate) particles can be produced more safely. In addition, it can be said that the use of the solvent having low toxicity is preferable from the viewpoint of environmental protection.

The amount of cellulose acetate in the cellulose acetate solution is determined according to the desired particle size and strength of cellulose (acetate) particles as the product. The desired particle size and strength vary depending on the use of the cellulose (acetate) particles and can be appropriately adjusted.

For example, with respect to 100% by weight of the cellulose acetate solution, the amount of cellulose acetate is preferably 1% by weight to 30% by weight, more preferably 2% by weight to 20% by weight, and particularly preferably 4% by weight to 12% by weight. By setting the content of cellulose acetate in the above range, particles having mechanical strength can be obtained. In addition, spherical particles are easily obtained.

As the amount of cellulose acetate in the cellulose acetate solution increases, while the viscosity of the solution increases, the solid content and the strength of the obtained cellulose (acetate) particles increase. However, when the content of cellulose acetate is too high, problems may occur in which the viscosity becomes too high and the operability deteriorates, or flake-shaped instead of spherical particles are precipitated. On the other hand, when the content of cellulose acetate is too low, the cellulose acetate may not be precipitated as particles or the mechanical strength of the cellulose (acetate) particles may be reduced.

In (a), the cellulose acetate is dissolved in the solvent preferably at a temperature of 25° C. to 100° C., more preferably 40° C. to 100° C., and particularly preferably 40° C. to 90° C. By setting the temperature to 25° C. or higher, the cellulose acetate can be promptly dissolved in the solvent. On the other hand, if the temperature is 100° C. or lower, the choice of the solvent to be used is wide, which is therefore preferable. Depending on the solvent used, it is preferable to adjust the temperature to not exceed the boiling point of the solvent. In addition, by setting the operating temperature to 25° C. to 100° C., there is an advantage that the particle size of the cellulose (acetate) particles can be easily controlled.

[Step (b)]

In (b), the cellulose acetate solution obtained in (a) and an aqueous medium were passed through a gap between an outer cylinder and an inner cylinder arranged coaxially inside the outer cylinder, and at least one of the outer cylinder and the inner cylinder is rotated. Accordingly, an emulsion in which cellulose acetate droplets are dispersed in the aqueous medium is obtained. The cellulose acetate solution and the aqueous medium may be separately introduced into a device from different inlets, or a coarse emulsion containing the cellulose acetate solution and the aqueous medium may be prepared in advance, and the coarse emulsion may be introduced into the device from a single inlet.

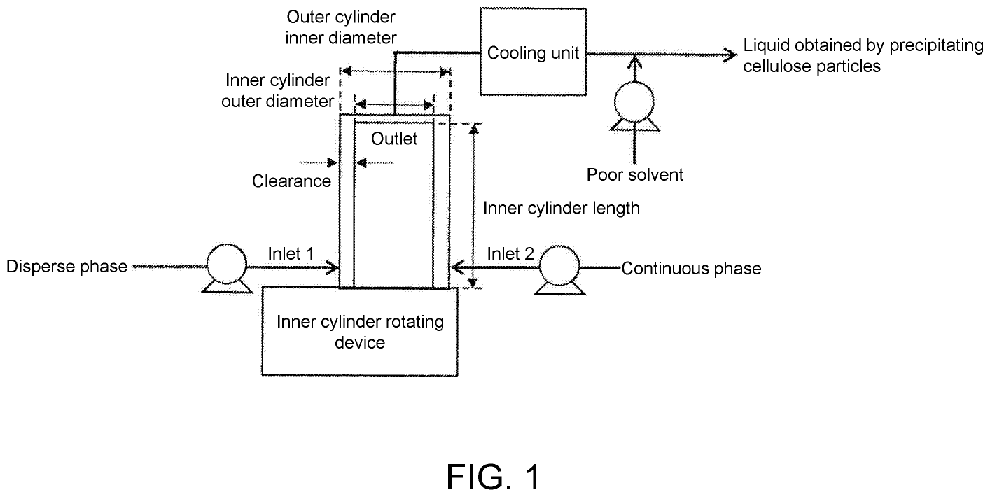

Moreover, please refer to FIG. 1 for an outline of a device including the outer cylinder and the inner cylinder, which is used in (b).

In (b), an inner cylinder outer diameter is not particularly limited, and is preferably 10 mm to 499.8 mm, more preferably 50 mm to 100 mm. An outer cylinder inner diameter is not particularly limited, and is preferably 10.2 mm to 500 mm, more preferably 50 mm to 100 mm. Since the inner cylinder is arranged inside the outer cylinder, the outer cylinder inner diameter is necessarily larger than the inner cylinder outer diameter.

In addition, a clearance (distance between the inner cylinder and the outer cylinder) is not particularly limited, and is preferably 0.1 mm to 10 mm, more preferably 0.5 mm to 5 mm.

The shapes of the inner cylinder and the outer cylinder are not particularly limited as long as they are coaxially arranged and do not hinder rotation. In addition, the respective lengths of the cylinders are not particularly limited, and are preferably 50 mm to 800 mm, more preferably 100 mm to 500 mm.

In (b), by rotating at least one of the outer cylinder and the inner cylinder, the solution existing in the gap therebetween is emulsified by a shearing force. In this specification, the cylinder rotation may be rotation of at least any one of the outer cylinder and the inner cylinder if the inner cylinder and the outer cylinder are relatively rotated, but is preferably rotation of only the inner cylinder considering safety. When both the inner cylinder and the outer cylinder are rotated, they are preferably rotated in opposite directions considering efficiency.

A rotational speed (ω) is not particularly limited, and is preferably 300 rpm to 3000 rpm, more preferably 500 rpm to 2000 rpm. As shown in examples to be described later, it is possible to control the particle size of the obtained cellulose (acetate) particles by changing the rotational speed.

In addition, emulsification time, that is, the time during which the cylinder is rotating while the cellulose acetate solution and the aqueous medium pass through the gap, is not particularly limited, and is preferably 0.01 second to 600 seconds, more preferably 0.1 second to 150 seconds.

In (b), temperatures of the cellulose acetate solution and the aqueous medium when the cellulose acetate solution and the aqueous medium pass through the gap between the outer cylinder and the inner cylinder are not particularly limited, and are preferably 20° C. to 90° C., more preferably 30° C. to 70° C. The above temperature range is preferable from the viewpoint of dispersibility of the cellulose acetate solution in the dispersion medium. In addition, the above temperature range is preferable because the form of the cellulose acetate solution in the dispersion medium can be kept spherical. On the other hand, when the operating temperature exceeds 100° C., it may approach a boiling point of the dispersion medium, which may be unfavorable depending on the dispersion medium used.

In step (b), generally, the cellulose acetate solution and the aqueous medium are supplied to the gap between the outer cylinder and the inner cylinder, emulsification is performed and then the emulsion is discharged. At this time, the passage through the gap between the outer cylinder and the inner cylinder is preferably performed a plurality of times. That is, the solution is repeatedly supplied to and discharged from the gap between the outer cylinder and the inner cylinder at least one of which is rotating. The number of repetitions is not particularly limited, and is preferably 1 to 10, more preferably 1 to 4.

In addition, the supply of the cellulose acetate solution and the aqueous medium to the gap between the outer cylinder and the inner cylinder may be performed while the solution and the aqueous medium are continuously circulated. Emulsification operation time, that is, the time during which the continuous circulation is operating, is not particularly limited, and is preferably 5 minutes to 120 minutes, more preferably 10 minutes to 60 minutes.

Furthermore, step (b) is preferably performed in multiple stages using a continuous stirred-tank reactor (CSTR).

In (b), the aqueous medium can be used without particular limitation as long as being a medium capable of dispersing the cellulose acetate solution obtained in (a) without mixing therewith. Specific examples thereof include water, an ethyl acetate-containing aqueous solution, or a cyclohexanone-containing aqueous solution, or the like, and an ethyl acetate-containing aqueous solution is more preferable. In the case of the ethyl acetate-containing aqueous solution, a total concentration of ethyl acetate is not particularly limited, and is preferably 0.01% by weight to 10% by weight, more preferably 3% by weight to 7% by weight.

Moreover, the aqueous medium may contain other components as long as the effects of the disclosure are not impaired.

In (b), one or more kinds of surfactants may be arbitrarily added to the aqueous medium. By addition of the surfactant, the cellulose acetate solution droplets can be kept more spherical, coalescence of the droplets can be prevented, and the particle size can be controlled. Any surfactant can be used without particular limitation, and an anionic surfactant, a nonionic surfactant and a silicone-based surfactant are preferable. Examples thereof include linear alkylbenzene sulfonate, linear alkyl sulfonate, sorbitan monooleate, polyethylene glycol, polyvinyl alcohol, an alkyl glycoside, a polyoxyethylene-methyl polysiloxane copolymer, and so on.

According to the type and/or amount of the surfactant used, droplet size of the cellulose acetate solution can also be controlled. For example, if it is desired to reduce the droplet size of the cellulose acetate solution, sorbitan monooleate is preferably added; if it is desired to increase the droplet size, a polyoxyethylene-methylpolysiloxane copolymer is preferably added. In addition, the amount of the surfactant added is preferably 0.03% by weight to 3% by weight, more preferably 0.05% by weight to 2.5% by weight, and particularly preferably 0.1% by weight to 2.0% by weight, with respect to the medium. Here, especially in a cellulose diacetate solution, if it is desired to increase the droplet size, the amount of the surfactant added may be reduced; if it is desired to reduce the droplet size, the amount of the surfactant added may be increased.

[Step (c)]

In (c), cellulose acetate particles are precipitated from the emulsion obtained in (b) above.

A means of precipitation includes, for example, cooling the emulsion and/or adding a poor solvent to the emulsion. Here, it is preferable to perform both the cooling of the emulsion and the addition of the poor solvent, and it is more preferable to add the poor solvent after cooling the emulsion.

When the emulsion is cooled, phase separation occurs, the cellulose acetate particles are precipitated, and the particle size of the obtained cellulose (acetate) particles can be controlled by adjusting cooling conditions.

A cooling temperature is not particularly limited. From the viewpoint of easily controlling the particle size and shape of the obtained cellulose (acetate) particles, the cooling temperature is preferably lower than the temperature of the emulsion and is preferably 0° C. to 50° C. In addition, the cooling temperature is more preferably 10° C. to 30° C., and particularly preferably 15° C. to 25° C. If the cooling temperature is higher than 0° C., the whole dispersion system may not freeze; if the cooling temperature is 50° C. or lower, the desired cooling effect can be achieved.

In addition, a temperature drop rate at this time is preferably 0.1° C./min to 10° C./min, more preferably 2° C./min to 5° C./min.

When the poor solvent is added to the emulsion, phase separation occurs, the cellulose acetate particles are precipitated, and the particle size of the obtained cellulose (acetate) particles can be controlled by adjusting addition conditions.

The poor solvent is not particularly limited as long as it has low solubility in cellulose acetate and its addition causes the precipitation of cellulose acetate particles. Specifically, for example, water, alcohols, glycols, esters, and a mixture thereof can be used. As the alcohols, lower alcohols are preferable, and alcohols having 1 to 3 carbon atoms are more preferable. Specific examples thereof include methanol, ethanol, 1-propanol, 2-propanol and so on. Examples of the glycols include ethylene glycol, propylene glycol, diethylene glycol, trimethylene glycol and so on. Examples of the esters include ethyl acetate, butyl acetate, ethyl lactate, ethyl butyrate and so on.

In the case where a mixed solvent is used as the poor solvent, the mixed solvent is preferably a mixture of water and the alcohols, a mixture of water and the glycols, or a mixture of water and the esters. The mixed solvent is more preferably a mixture of water and the alcohols, and is particularly preferably a mixture of water and methanol, a mixture of water and ethanol, or a mixture of water and 2-propanol. A mixing ratio of these mixtures is preferably 0.01% by weight to 30% by weight, more preferably 10% by weight to 20% by weight in terms of the concentration of alcohols.

The precipitated cellulose acetate particles are separated by any method known to those skilled in the art. The separation can be performed, for example, by filtration or the like.

In the case of producing cellulose particles, the cellulose acetate particles are subjected to the following (d).

[Step (d)]

In (d), the cellulose acetate particles precipitated in (c) above are saponified. Accordingly, an ester portion of the cellulose acetate is hydrolyzed and cellulose particles are obtained.

The saponification can be performed by a method known to those skilled in the art, and can be performed using, for example, an alkali and an alcohol aqueous solution. As the alkali, for example, a sodium hydroxide aqueous solution, a potassium hydroxide aqueous solution or the like is preferably used. In addition, as the alcohol in the alcohol aqueous solution, a lower alcohol is preferable. For example, methanol and ethanol are more preferable. Specifically, the saponification can be performed by stirring the cellulose acetate particles obtained in (c) in, for example, a mixture of the alkali and the alcohol aqueous solution, for a certain period of time.

The cellulose acetate particles obtained in (c) are desired to be washed before being saponified. A solution used in the washing is not particularly limited as long as being capable of washing the cellulose acetate particles without destroying their structure. For example, methanol, water or the like can be used.

When cellulose diacetate is used as a raw material in the method of the disclosure, it is possible to select a solvent having low toxicity as a solvent in preparing a cellulose solution. Therefore, the cellulose (acetate) particles can be produced by a method which is safer and is preferable from the viewpoint of environmental protection.

In addition, by using the emulsification method performed by cylinder rotation, the productivity is remarkably improved, and cellulose (acetate) particles having a uniform particle size distribution, specifically, a small CV (50% or less), can be obtained as compared with a conventional production method. In addition, it has also been confirmed that the space-time yield is improved as compared with the conventional manufacturing method, for example, the method of Patent Document 9.

Furthermore, according to the production method of the disclosure, the particle size of the obtained cellulose (acetate) particles can be arbitrarily controlled or the particle size distribution can be controlled. Therefore, cellulose (acetate) particles having an optimum particle size for each use can be obtained. According to the method according to the embodiment of the disclosure, cellulose (acetate) particles having a wide particle size range can be obtained. That is, it is possible to obtain cellulose (acetate) particles having a particle size falling in any of a small size region, a medium size region, and a large size region.

The particle size of the cellulose (acetate) particles can be easily controlled by changing various conditions in the method of the disclosure. For example, the particle size can be controlled by the operating temperature of each step, the amount of the cellulose diacetate raw material used, the types of the solvent and the poor solvent used, the type and the amount of the surfactant added, and so on.

Specifically, in (a), the higher the cellulose acetate concentration in the cellulose acetate solution, the larger the particle size and coefficient of variation (CV) of the obtained cellulose (acetate) particles respectively tend to be.

In (b), the higher the rotational speed, the smaller the particle size and CV of the obtained cellulose (acetate) particles respectively tend to be.

In (b), the larger the clearance, the larger the particle size and CV of the obtained cellulose (acetate) particles respectively tend to be.

In (b), the longer the emulsification time (time of rotation of the outer cylinder and/or inner cylinder), the smaller the particle size and CV of the obtained cellulose (acetate) particles respectively tend to be.

In addition, when the amount of the surfactant used is increased, the particle size of the obtained cellulose (acetate) particles tends to decrease; in contrast, if the amount of the surfactant is reduced, the particle size of the obtained cellulose (acetate) particles tends to increase. In addition, it is possible to change the particle size distribution of the obtained cellulose (acetate) particles by changing the type of the surfactant used.

The particle size of the cellulose (acetate) particles is not particularly limited as long as the object of the disclosure is not impaired. For example, from the viewpoint of being usable as an adsorbent for various substances, the cellulose (acetate) particles preferably have a particle size of 1 μm to 2 mm, more preferably 20 μm to 1 mm, and particularly preferably 35 μm to 600 μm.

In addition, the cellulose (acetate) particles can be used as an additive to a cosmetic, or a daily commodity such as a hygiene product, a sanitary product, a laundry detergent, an oral care product, a toiletry product or the like. In that case, the particle size of the cellulose (acetate) particles is preferably 1 μm to 30 μm, more preferably 2 μm to 20 μm, and particularly preferably 5 μm to 15 μm. If the particle size is 1 μm to 30 μm, the cellulose (acetate) particles can be used for various purposes such as cosmetics or various daily commodities. In particular, when a smooth feel is desired for the cellulose (acetate) particles as a cosmetic, the particle size is preferably 5 μm to 15 μm because slipperiness is improved.

Further, depending on the purpose, a plurality of cellulose (acetate) particles having different particle sizes may be mixed for use.

The particle size of the cellulose (acetate) particles can be calculated, for example, by measuring the particle size distribution using a particle size distribution measurement device (“Laser Scattering Particle Size Distribution Analyzer Partica LA-950” made by HORIBA).

The cellulose (acetate) particles that can be produced by the production method of the disclosure can be provided for various uses in either a modified form or an unmodified form.

For example, the cellulose (acetate) particles can be used in separation and purification of various substances. For example, the cellulose (acetate) particles can be used to fractionate substances having different molecular sizes in the gel filtration method such as size exclusion chromatography or the like. At this time, the cellulose (acetate) particles obtained by the disclosure may be applied as they are, or may be applied in a form of being further modified with a substituent or having undergone a crosslinking reaction.

In addition, by adding a ligand to at least some of reactive functional groups of the cellulose (acetate) particles of the disclosure, an adsorbent capable of adsorbing various substances can be easily obtained. For example, the cellulose (acetate) particles may be used in a virus adsorbent against an influenza virus or hepatitis B or the like, an adsorbent for antibody drug purification, a low-density lipoprotein (LDL) cholesterol adsorbent, or the like. According to the method according to the embodiment of the disclosure, as described above, since the particle size of the cellulose (acetate) particles can be easily controlled in a wide range, it is possible to appropriately produce an adsorbent having excellent adsorption properties and less nonspecific adsorption depending on the use.

Specifically, by subjecting at least some of hydroxyl groups contained in the cellulose particles of the disclosure to a sulfation treatment and introducing a sulfate group (—OSO₃H) into the cellulose particles, a chromatography packing material suitable for separation or purification of a protein such as lysozyme, an immunoglobulin, a blood coagulation factor or the like can be provided.

A method for introducing a sulfate group into the cellulose particles of the disclosure, that is, a method for obtaining sulfated cellulose particles, is not particularly limited, and can be performed, for example, as follows.

First, a sulfation agent is prepared in a reaction vessel. The sulfation agent used in the disclosure is not particularly limited as long as being capable of reacting with the hydroxyl group in the cellulose particles and introducing the sulfate group into the cellulose particles. Examples of such a sulfation agent include a chlorosulfonic acid-pyridine complex, piperidine-N-sulfuric acid, a sulfuric anhydride-dimethylformamide complex, a sulfur trioxide-pyridine complex, a sulfur trioxide-trimethylamine complex, a sulfuric acid-trimethylamine complex and so on. The amount of the sulfation agent used may be arbitrarily selected depending on a target introduction rate of the sulfate group and reaction conditions. For example, the sulfation agent is suitably used in an amount of 0.001 to 1 equivalent with respect to the hydroxyl group in the cellulose particles.

Next, the cellulose particles that have been dried are added to the sulfation agent to carry out a sulfation reaction. The reaction temperature and time vary depending on the type of the solvent or the sulfation agent. In an inert gas, the reaction is generally carried out at 0° C. to 100° C., preferably at 20° C. to 85° C., for preferably 0.5 hour to 24 hours, more preferably 0.5 hour to 10 hours.

After the reaction is completed, an alkali aqueous solution, for example, a sodium hydroxide aqueous solution, may be added to the reaction mixture for neutralization.

After that, by filtration or centrifugal separation of the obtained reaction mixture, a product is collected, and is washed with water until becoming neutral, and the target sulfated cellulose particles can be obtained. The amount of the sulfate group introduced into the sulfated cellulose particles can be adjusted by changing the amount of the sulfation agent used, and may be appropriately determined depending on the use of the chromatography packing material, or the like.

In addition, by subjecting at least some of the hydroxyl groups in the cellulose particles of the disclosure to a sulfonation treatment and introducing a sulfonic acid group-containing group into the particles, a strong cation ion exchange chromatography packing material suitable for separation or purification of a protein such as an immunoglobulin, lysozyme or the like can also be provided.

The sulfonic acid group-containing group that can be introduced into the cellulose particles of the disclosure is not particularly limited as long as being a hydrocarbon group containing a sulfonic acid group (—SO₃H). The hydrogen atom contained in the sulfonic acid group-containing group may further be replaced by a substituent such as a hydroxyl group, a halogen atom, epoxy group or the like. Among them, the sulfonic acid group-containing group to be introduced is preferably a sulfoalkyl group having 1 to 5 carbon atoms, which may have a substituent.

A method for introducing the sulfonic acid group-containing group into the cellulose particles of the disclosure is not particularly limited as long as being generally used in a sulfonation treatment of polysaccharides. For example, a method may be mentioned in which the cellulose particles of the disclosure are treated using a sulfonation agent, such as haloalkane sulfonate such as sodium 3-chloro-2-hydroxypropanesulfonate, sodium 3-bromopropane sulfonate or the like, or sulfonic acid having an epoxy group, such as 1,4-butanesultone, 1,3-propanesultone or 1,2-epoxyethanesulfonic acid or the like.

The amount of the sulfonic acid group-containing group introduced into the sulfonated cellulose particles can be adjusted by changing the amount of the sulfonation agent or alkali used, and may be appropriately determined depending on the use of the chromatography packing material.

The sulfonation treatment of the cellulose particles can be performed with reference to Japanese Patent Laid-open No. 2001-302702 or Japanese Patent Laid-open No. H9-235301. By appropriately changing the design of experimental conditions, the target sulfonic acid group-containing group can be introduced in a target amount.

As described above, according to one aspect of the disclosure, a chromatography packing material or an adsorbent is provided containing the cellulose particles obtained by the disclosure or the cellulose particles that are modified. The chromatography packing material or the adsorbent according to the disclosure can be used particularly for separating and purifying a protein such as lysozyme, an immunoglobulin, a blood coagulation factor or the like, and particles of a virus such as an influenza virus, hepatitis B or the like.

In addition, the cellulose (acetate) particles that can be produced by the production method of the disclosure, in either the modified form or the unmodified form, can be used as an additive to a cosmetic or a daily commodity or the like. In particular, cellulose is in high demand as a natural material because of its biodegradability, and can contribute to a reduction of environmental load as a substitute for petroleum-derived fine particles widely used at present such as acrylic fine particles, styrene fine particles, urethane fine particles, ethylene fine particles, melamine fine particles and so on.

When the cellulose (acetate) particles that can be produced by the production method of the disclosure are used as a cosmetic, depending on the purpose of use, a conventional additive, such as a stabilizer such as an alcohol, methyl cellulose, carboxymethyl cellulose (CMC) or the like, a surfactant, a fragrance, a preservative, an antioxidant, collagen, an ultraviolet absorber, a mucopolysaccharide, a binder, an extender or the like, may be contained. A cosmetic containing these is used in a liquid form such as a lotion, an emulsion, a cream, a gel or the like, or a solid form such as a powder, a granule, a paste, a molded product or the like. For example, the cosmetic can be used as a foundation, a lip balm, an eyeshadow, an antiperspirant, a deodorant, a skin toner, a lotion, a milky lotion, a beauty essence, a hand cream, a body cream, a sunscreen, a toilet soap, a face wash, a peeling agent, an exfoliator, a pack agent, a body powder, or the like.

The cellulose (acetate) particles of the disclosure can be used in a wide range of fields as a component of a cosmetic, and the amount thereof added may be appropriately adjusted depending on the purpose of use. For example, when used as a foundation, the amount thereof added is preferably 1% by weight to 15% by weight; when used as a lip balm, the amount thereof added is preferably about 3% by weight; when used as an eyeshadow, the amount thereof added is preferably 3% by weight to 5% by weight; when used as an antiperspirant or deodorant, the amount thereof added is preferably about 7% by weight; and when used as a skin toner, lotion or milky lotion, the amount thereof added is preferably about 1% by weight to 5% by weight.

The cellulose (acetate) particles that can be produced by the production method of the disclosure can be used for the purpose of improving the usability of a daily commodity. Examples of the daily commodity include a hygiene product such as a wet tissue, a paper diaper or the like, a sanitary product such as a napkin or the like, a laundry detergent such as a fabric softener or the like, an oral care product such as a toothpaste, a mouthwash, a mouth freshener or the like, a toiletry product such as a hand soap, a body soap or the like, a hair care product such as a shampoo, a conditioner, a hair color, a hair spray, a hair wax, a hair restorer or the like, a shaving product and so on.

### EXAMPLES

Hereinafter, the disclosure will be described in more detail with reference to examples, but the disclosure is not limited thereto.

### Example 1

Step (a)

0.2 g of cellulose diacetate (L-20, made by Daicel, degree of acetylation: 55% to 56%) was added to 4.8 g of cyclohexanone and stirred. Further, by heating and stirring at 60° C. for 3 hours or more, the cellulose diacetate was dissolved, and a cellulose acetate solution having a cellulose diacetate concentration of 4% by weight was prepared and used as a disperse phase.

Step (b)

0.1 g of sodium dodecylbenzenesulfonate as a surfactant and 3.5 g of cyclohexanone were added to 50 g of pure water and stirred. Further, an aqueous medium was prepared by heating to 60° C., and was used as a continuous phase.

The disperse phase obtained above was preheated to 60° C. in advance and was supplied to an inlet 1 (inlet 1 in FIG. 1) of an inner cylinder rotating device (having an inner cylinder outer diameter of 78 mm, an inner cylinder length of 215 mm, an outer cylinder inner diameter of 80 mm, and a clearance of 1 mm, made by Tipton Corp.) at 1 mL/min using a syringe pump (high pressure micro feeder JP-H, made by Furue Science Co., Ltd.), the continuous phase obtained above was preheated to 60° C. in advance and was supplied to an inlet 2 (inlet 2 in FIG. 1) of the inner cylinder rotating device at 10 mL/min using a plunger pump (NP-KX-840, made by Nihon Seimitsu Kagaku Co., Ltd.), and emulsification was performed at an inner cylinder rotational speed of 2000 rpm for 138 seconds (emulsification time), with the result that an oil-in-water emulsion was obtained.

Step (c)

Subsequently, by cooling the oil-in-water emulsion to 5° C., supplying the emulsion and pure water as a poor solvent to a double-pipe merger at 10 mL/min and precipitating cellulose diacetate, a cellulose diacetate particle slurry was obtained. After that, the above dispersion was subjected to solid-liquid separation by a centrifugal separation method and a filtration method, and the obtained cellulose diacetate particles were sufficiently washed with a large amount of water. Then, the spherical cellulose diacetate particles after washing were filtered and cellulose diacetate particles as the product were obtained.

Step (d)

In addition, the obtained cellulose diacetate particles were added to a mixture of 55% by weight methanol aqueous solution (7 parts by weight) and 20% by weight sodium hydroxide aqueous solution (3.5 parts by weight) and stirred at 35° C. for 20 hours, thereby saponifying the cellulose diacetate particles. As a result, cellulose particles as the final product were obtained.

### Test Example 1: Measurement of Particle Size Distribution

The cellulose diacetate particles obtained in Example 1 were measured for particle size distribution. An obtained median diameter was defined as a particle size, and a CV was calculated from the values of the particle size and a standard deviation by the following equation. A device used in the measurement was as follows.

Device: Laser Scattering Particle Size Distribution Analyzer Partica LA-95052 (made by HORIBA)

CV [%]=(σ/D)×100 (σ: standard deviation; D: particle size)

### Example 2

Cellulose diacetate particles were obtained in the same manner as in Example 1 except that the clearance was changed.

The results of Examples 1 and 2 are shown in Table 1 below. From Table 1, it can be said that the larger the clearance, the larger the particle diameter and CV of the obtained cellulose diacetate particles tend to be.

TABLE 1

| | Clearance | Particle size | CV |
|---|---|---|---|
| | [mm] | [μm] | [%] |
| Example 1 | 1 | 4.0 | 37.1 |
| Example 2 | 2 | 4.7 | 48.1 |

### Examples 3 to 7

Cellulose diacetate particles were obtained in the same manner as in Example 1 except that the inner cylinder rotational speed and the emulsification time were changed.

The results of Examples 3 to 7 are shown in Table 2 below. From Table 2, it can be said that the higher the inner cylinder rotational speed, the smaller the particle size and CV of the obtained cellulose diacetate particles tend to be. It can also be said that the longer the emulsification time, the smaller the particle size of the obtained cellulose diacetate particles tends to be.

TABLE 2

| | Inner cylinder | Emulsification | Particle | |
|---|---|---|---|---|
| | rotational speed | time | size | CV |
| | [rpm] | [sec] | [μm] | [%] |
| Example 3 | 2000 | 3 | 5.5 | 38.4 |
| Example 4 | 1500 | 3 | 12.1 | 42.6 |
| Example 5 | 1000 | 3 | 29.7 | 46.5 |
| Example 6 | 500 | 3 | 64.1 | 65.2 |
| Example 7 | 1000 | 136 | 6.6 | 42.8 |

### Examples 8 to 10

Cellulose diacetate particles were obtained in the same manner as in Example 1 except that the cellulose acetate concentration in the cellulose diacetate solution and the emulsification time were changed.

The results of Examples 8 to 10 are shown in Table 3 below. From Table 3, it can be said that the higher the cellulose diacetate concentration, the larger the particle size and CV of the obtained cellulose diacetate particles tend to be.

TABLE 3

| | Cellulose | | | |
|---|---|---|---|---|
| | diacetate | Emulsification | Particle | |
| | concentration | time | size | CV |
| | [% by weight] | [sec] | [μm] | [%] |
| Example 8 | 4 | 3 | 5.5 | 38.4 |
| Example 9 | 6 | 3 | 11.9 | 57.6 |
| Example 10 | 8 | 3 | 11.0 | 86.6 |

### Example 11

Step (a) 2.0 g of cellulose diacetate (L-20, made by Daicel, degree of acetylation: 55% to 56%) was added to 48.0 g of cyclohexanone and stirred. Further, by heating and stirring at 60° C. for 3 hours or more, the cellulose diacetate was dissolved, and a cellulose acetate solution having a cellulose diacetate concentration of 4% by weight was prepared and used as a disperse phase.

Step (b)

The cellulose acetate solution (disperse phase) was poured into 500 g of 60° C. pure water as an aqueous medium (continuous phase) containing 1.0 g of sodium dodecylbenzenesulfonate as a surfactant and 32.5 g of cyclohexanone, the resultant was stirred at a rotational speed of 100 rpm for 60 minutes and a coarse emulsion was prepared.

The coarse emulsion obtained above was preheated to 60° C. in advance and was supplied to the inlet 1 of an inner cylinder rotating device (having an inner cylinder outer diameter of 78 mm, an inner cylinder length of 215 mm, an outer cylinder inner diameter of 80 mm, and a clearance of 1 mm, made by Tipton Corp.) at 11 mL/min using a plunger pump (NP-LX-300, made by Nihon Seimitsu Kagaku Co., Ltd.), and emulsification was performed at an inner cylinder rotational speed of 2000 rpm for 138 seconds (emulsification time). The obtained emulsion was further subjected to a continuous circulation operation for 10 minutes (emulsification operation time) by the plunger pump, with the result that an oil-in-water emulsion was obtained.

Step (c)

Subsequently, 500 mL of pure water as a poor solvent was added dropwise to the oil-in-water emulsion. As a result, cellulose diacetate was precipitated, and spherical cellulose diacetate particles were obtained. The subsequent washing and particle size distribution measurement were performed in the same manner as in Example 1.

### Example 12

Cellulose diacetate particles were obtained in the same manner as in Example 11 except that the emulsification operation time was changed to 60 minutes.

The results of Examples 11 to 12 are shown in Table 4. From Table 4, it can be said that the longer the emulsification operation time, the smaller the particle size and CV of the obtained particles tend to be.

TABLE 4

| | Emulsification | Particle | |
|---|---|---|---|
| | operation time | size | CV |
| | [min] | [μm] | [%] |
| Example 11 | 10 | 6.3 | 43.7 |
| Example 12 | 60 | 4.3 | 41.0 |

### Example 13

Step (a) 1 g of cellulose diacetate (L-20, made by Daicel, degree of acetylation: 55% to 56%) was added to 7.2 g of ethyl acetate and 1.8 g of acetone and stirred. Further, by heating and stirring at 50° C. for 3 hours or more, the cellulose diacetate was dissolved, and a cellulose acetate solution having a cellulose diacetate concentration of 10% by weight was prepared and used as a disperse phase.

Step (b)

0.1 g of sodium dodecylbenzenesulfonate as a surfactant and 3.5 g of ethyl acetate were added to 46.5 g of pure water and stirred. Further, an aqueous medium was prepared by heating to 50° C., and was used as a continuous phase.

The disperse phase obtained above was preheated to 50° C. in advance and was supplied to the inlet 10 (inlet 1 in FIG. 1) of an inner cylinder rotating device (having an inner cylinder outer diameter of 78 mm, an inner cylinder length of 215 mm, an outer cylinder inner diameter of 80 mm, and a clearance of 1 mm, made by Tipton Corp.) at 10 mL/min using a diaphragm pump (XPL-03-6T6X-MVX, made by TACMINA CORPORATION), the continuous phase obtained above was preheated to 50° C. in advance and was supplied to the inlet 2 (inlet 2 in FIG. 1) of the inner cylinder rotating device at 50 mL/min using a plunger pump (NP-KX-840, made by Nihon Seimitsu Kagaku Co., Ltd.), and emulsification was performed at an inner cylinder rotational speed of 1500 rpm for 53 seconds (emulsification time), with the result that an oil-in-water emulsion was obtained.

Step (c)

Subsequently, by cooling the oil-in-water emulsion to 25° C., supplying the emulsion and pure water as a poor solvent to a double-pipe merger at 80 mL/min and precipitating cellulose diacetate, a cellulose diacetate particle slurry was obtained. The subsequent washing and particle size distribution measurement were performed in the same manner as in Example 1.

### Examples 14 to 16

Cellulose diacetate particles were obtained in the same manner as in Example 13 except that the cellulose diacetate concentration in the cellulose acetate solution and the inner cylinder rotational speed were changed.

The results of Examples 13 to 16 are shown in Table 5 below. As is clear from Table 5, the higher the cellulose diacetate concentration, the larger the particle size and CV of the obtained cellulose diacetate particles tend to be. It can also be said that the higher the inner cylinder rotational speed, the smaller the particle size and CV of the obtained cellulose diacetate particles tend to be.

TABLE 5

| | Cellulose diacetate | Inner cylinder | Particle | |
|---|---|---|---|---|
| | concentration | rotational speed | size | CV |
| | [% by weight] | [rpm] | [μm] | [%] |
| Example 13 | 10 | 1500 | 7.9 | 53.0 |
| Example 14 | 4 | 1500 | 4.3 | 37.8 |
| Example 15 | 10 | 2000 | 6.6 | 53.1 |
| Example 16 | 10 | 1000 | 17.2 | 90.6 |

### Comparative Example 1

2.0 g of cellulose diacetate (L-20, made by Daicel, degree of acetylation: 55% to 56%) was added to 48.0 g of cyclohexanone and stirred. Further, by heating and stirring at 60° C. for 3 hours or more, the cellulose diacetate was dissolved, and a cellulose acetate solution (disperse phase) having a cellulose diacetate concentration of 4% by weight was prepared. The solution was poured into 500 g of 60° C. pure water as an aqueous medium (continuous phase) containing 1.0 g of sodium dodecylbenzenesulfonate as a surfactant and 32.5 g of cyclohexanone, the resultant was stirred at a rotational speed of 100 rpm for 60 minutes and an oil-in-water coarse emulsion was obtained.

Subsequently, the oil-in-water coarse emulsion was cooled to 5° C., and 380 mL of pure water as a poor solvent was added dropwise thereto. As a result, cellulose diacetate was precipitated, and spherical cellulose diacetate particles were obtained. The subsequent washing and particle size distribution measurement were performed in the same manner as in Example 1.

The results of Comparative Example 1 are shown in Table 6 below in comparison with Examples 11 and 12.

TABLE 6

| | Emulsification | Particle | |
|---|---|---|---|
| | operation time | size | CV |
| | [min] | [μm] | [%] |
| Example 11 | 10 | 6.3 | 43.7 |
| Example 12 | 60 | 4.3 | 41.0 |
| Comparative | No emulsification | 10.2 | 52.4 |
| Example 1 | operation | | |

It is clear that the cellulose diacetate particles obtained from the coarse emulsion (without emulsification operation) that does not undergo the emulsification method performed by cylinder rotation in which at least one of the outer cylinder and the inner cylinder is rotated have increased particle size and CV. If the CV is large, that is, if the particle size distribution is broad, during a subsequent step of washing the particles, it is expected that filtration time or required pressure will increase due to filtration work and so on, and there is further a concern that the particles may be deformed or damaged due to the increased filtration pressure. From such a viewpoint, it is clear that the production method of the disclosure leads to an improvement in productivity based on the subsequent step.

### INDUSTRIAL APPLICABILITY

According to the disclosure, cellulose particles can be produced in a manner enabling control of particle size and particle size distribution and increasing the productivity. The cellulose particles obtained by the production method of the disclosure are applicable to various uses such as chromatography or additives to cosmetics and daily commodities and are therefore very useful industrially.

What is claimed is:

1. A method for producing cellulose acetate particles, comprising:
(a) dissolving cellulose acetate in an organic solvent and preparing a cellulose acetate solution;
(b) obtaining an emulsion by passing the cellulose acetate solution and an aqueous medium through a gap between an outer cylinder and an inner cylinder arranged coaxially inside the outer cylinder and rotating at least one of the outer cylinder and the inner cylinder; and
(c) precipitating cellulose acetate particles from the emulsion.

2. The method for producing cellulose acetate particles according to claim 1, wherein in (a), the organic solvent is ethyl acetate, a mixed solvent of ethyl acetate and acetone, or cyclohexanone.

3. The method for producing cellulose acetate particles according to claim 1, wherein in (a), the cellulose acetate is cellulose diacetate having a degree of acetylation of 45% to 57%.

4. The method for producing cellulose acetate particles according to claim 1, wherein in (c), the cellulose acetate particles are precipitated by at least one of cooling of the emulsion and addition of a poor solvent to the emulsion.

5. The method for producing cellulose acetate particles according to claim 4, wherein the poor solvent is water, alcohols, glycols, esters, or a mixture thereof.

6. The method for producing cellulose acetate particles according to claim 1, wherein in (b), the passage through the gap between the outer cylinder and the inner cylinder is performed a plurality of times.

7. The method for producing cellulose acetate particles according to a claim 1, wherein in (b), a particle size of the cellulose acetate particles is controlled by changing a rotational speed of at least one of the outer cylinder and the inner cylinder.

8. The method for producing cellulose acetate particles according to claim 1, wherein in (b), the aqueous medium is water, an ethyl acetate-containing aqueous solution, or a cyclohexanone-containing aqueous solution.

9. The method for producing cellulose acetate particles according to claim 1, wherein in (a), the cellulose acetate solution is prepared so as to contain 1% to 30% by weight of cellulose acetate with respect to the whole of the cellulose acetate solution.

10. A method for producing cellulose particles, comprising:
producing cellulose acetate particles by the method for producing cellulose acetate particles according to claim 1; and
(d) saponifying the cellulose acetate particles.

11. Cellulose acetate particles produced by the method according to claim 1.

12. A chromatography packing material, containing the cellulose acetate particles according to claim 11, wherein the cellulose acetate particles are modified or unmodified.

13. A cosmetic, containing the cellulose acetate particles according to claim 11, wherein the cellulose acetate particles are modified or unmodified.

14. A daily commodity, containing the cellulose acetate particles according to claim 11, wherein the cellulose acetate particles are modified or unmodified.

15. Cellulose particles produced by the method according to claim 10.

16. A chromatography packing material, containing the cellulose particles according to claim 15, wherein the cellulose particles are modified or unmodified.

17. A cosmetic, containing the cellulose particles according to claim 15, wherein the cellulose particles are modified or unmodified.

18. A daily commodity, containing the cellulose particles according to claim 15, wherein the cellulose particles are modified or unmodified.
